(19) United States
(12) Patent Application Publication
(10) Pub. No.: US 2013/0090571 A1
(43) Pub. Date: Apr. 11, 2013

(54) METHODS AND SYSTEMS FOR MONITORING AND PREVENTING PRESSURE ULCERS

(71) Applicants: Mehrdad Nourani, Plano, TX (US); Matthew Q. Pompeo, Dallas, TX (US); Lakshman S. Tamil, Plano, TX (US); Sarah Ostadabbas, Dallas, TX (US); Rasoul Yousefi, Dallas, TX (US)

(72) Inventors: Mehrdad Nourani, Plano, TX (US); Matthew Q. Pompeo, Dallas, TX (US); Lakshman S. Tamil, Plano, TX (US); Sarah Ostadabbas, Dallas, TX (US); Rasoul Yousefi, Dallas, TX (US)

(73) Assignee: The Board of Regents of the University of Texas System, Austin, TX (US)

(21) Appl. No.: 13/267,513

(22) Filed: Oct. 6, 2011

(51) Int. Cl. A61B 5/103

(52) U.S. Cl. 600/587

(57) ABSTRACT

The present invention relates generally to the prevention and treatment of pressure ulcers and a platform for monitoring, prevention and management of pressure ulcer using a pressure mapping system that records a patient's bed posture, tracks different limbs along with associated statistical pressure image data and produces a summary report for care givers after data analysis and risk assessment. The methodology allows care givers to utilize the stress data and schedule the repositioning of the patient more effectively. The invention relates to creation and using algorithms and analytics for monitoring, prevention and management of pressure ulcers which include time-stamped whole-body pressure distribution data collection and profiling; posture classification, limb detection and tracking; quality of turn and risk assessment; turning schedule and nursing staff utilization for pressure ulcer management; and patient status reporting system customized for caregivers.

### BACKGROUND OF THE INVENTION

This invention relates generally to sensing and monitoring one or more conditions related to the health of the human body, such as the development, prevention and treatment of pressure ulcers. The present invention more particularly relates to a system and method of monitoring one or more pressure sensitive areas of a human body. Although aspects of the present invention have application with regard to other human body conditions, the invention will be specifically described in the context of pressure that contributes to the development of external skin ulcers (e.g., decubitus ulcers). In this context, the invention also relates to computer-implemented pressure ulcer monitoring, prevention and management methods.

Pressure ulcers (PUs), also known as bedsores, develop mostly at the boney prominences of the body (e.g., heel, elbow, shoulders, ankles, sacrum). Currently, PU prevention is one of the greatest challenges facing caregivers, hospitals, and long term care facilities. PUs occur most frequently in institutionalized, community-dwellings and nursing homes for older adults, where there are serious problems that can lead to sepsis and death. In nursing homes, PUs represent a significant problem for residents (in terms of morbidity, pain, and reduced quality of life) and for facilities (in terms of staffing and costs of care). Once a PU develops, it is costly and extremely difficult to heal. They are very resistant to known medical therapy and, unlike acute wounds, PUs do not proceed through an orderly and timely process of healing to reduce anatomical or functional integrity.

Groups known to have a high risk of developing PUs include bedridden patients, wheelchair-bound individuals, frail elderly persons with no or limited mobility, as well as individuals with diabetes, poor nutrition, and chronic blood-flow diseases. Pressure ulcers represent an enormous burden on our health care system and an enormous problem for health care providers. In 1990, a large epidemiologic study reported that the 1-year incidence of PU development in nursing homes was 13.2%; a systematic review reported that in U.S. the prevalence ranged from 7% to 23%. In hospitalized patients, the prevalence ranges from about 3% to 11% (approximately 1.5-3.0 million patients in the United States). Pressure ulcers result in both an increased length of hospital stay and increased hospital costs. The current cost to our health care system resulting from PUs is more than $1.2 billion annually. Once developed, PUs represent an acute health condition that results in increased costs and suffering over many months and even years. Effective ulcer prevention and early detection will greatly reduce patient suffering/discomfort.

Pressure ulcers can develop quickly and are often very difficult to treat. They are painful and can lead to life threatening complications. Once developed, pressure ulcers increase hospital stay costs, imposing an enormous burden on our health care system. Despite considerable attempts to prevent pressure ulcers, prevalence figures remain unacceptably high. In 2009, the National Center of Health Statistics (NCHS) reported that more than 10% of the nursing home residents had developed a pressure ulcer. According to the Agency for Healthcare Research and Quality (AHRQ), among hospitalizations involving pressure ulcers as a primary diagnosis, about 1 in 25 admissions ended in death.

Early detection of any compromised skin area is the first and the most important step in preventing ulcers. The most effective care for an at-risk patient is to relieve the pressure. A common practice in hospitals is repositioning bed-bound patients every two hours. However, this fixed schedule doesn't take into account the patient's physiological state and clinical history. Studies have shown the risk of pressure ulcer development is influenced by several factors such as blood pressure, infection, disease conditions, age, sex and even fragile skin and nutrition. Chronic diseases including diabetes, vascular disease, and nervous system disabilities affecting mobility, can also speed up the pressure ulcer formation. Since each patient has a different risk level, some expectedly need more frequent pressure relief than others.

Given growing nursing shortage and escalating demands on the nursing staff makes it increasingly difficult to provide the same level of service to all of the patients. In 2000, the shortage of nurses was estimated at 6%. This shortage is expected to grow to 20% by 2015 and, if not addressed, to 29% by 2020. Therefore, efficient prevention planning base on need in the context of pressure ulcer alleviates the growing nursing shortage, and decreases the pressure ulcer formation incidents in the hospitals, thus reducing the resulting treatment costs.

Currently, the early diagnosis of a PU is conducted using visual and tactile investigation of the skin. The standard tactile tool used clinically is the blanch test. The blanch test involves applying gentle pressure to the skin to observe the whitening or blanching of the skin. A blanching area of reddened skin indicates healthy tissue structure and perfusion. Portable devices that measure non-blanchable erythema or the microcirculation properties of the skin related to pressure ulcer development are known. Staying in a fixed posture for a long time is known to cause pressure ulcers in stressed tissues. Continuous visual monitoring is not a practical option. Automatic posture detections have been neither accurate nor fast enough so far. To achieve a better performance, some researchers have used video cameras, which is not a preferred method due to the privacy concerns. Advancement in pressure sensing technology has provided opportunity to have pressure measurement in larger area with high resolution and low costs. There is a need for a high accuracy processing unit, for automated posture detection, limb detection/tracking and risk assessment which is capable of being used with current and future commercial pressure mapping systems. This processing algorithm should pave the road toward future usage of pressure mapping system.

The present invention is directed to a monitoring platform, using commercial (e.g. sensor mat) or custom-made (e.g. body-mounted sensor patches) pressure mapping system, that records patient's bed posture and tracks different limbs along with associated statistical pressure image data. Turning the patient every two hours, as hospital staffs are traditionally advised, is neither efficient nor practical. The methodology allows care givers to utilize the postures/limbs stress data and schedule the repositioning of the patient more effectively. It also allows continuous risk assessment and provides related information for creating an efficient monitoring and healing/treatment plan. An embodiment of the invention is directed to the use of algorithms and analytics for monitoring, prevention and management of pressure ulcers. Another embodiment of the invention provides a proposed bed architecture that can utilize the analyzed information and achieve the pressure distributions associated with turning a patient using a lesser, although significant, amount of rotational motion than manual turning conventionally done by caregivers.

### SUMMARY OF THE INVENTION

An embodiment of the invention is directed to a method of monitoring pressure applied to a pressure sensitive area of a human body, the method comprising: contacting a pressure sensor with the pressure sensitive area of the human body (for example using a pressure mat or body-mounted pressure sensor patches); determining body posture; tracking and determining the position of limbs of the human body; collecting data from the pressure sensor; extracting features from the collected data; training a model by using the extracted features; and determining the level of risk associated with the development of a pressure ulcer.

Another embodiment of the invention is directed to a system for monitoring pressure applied to a pressure sensitive area of a human body (some of which are shown in FIGS. 1A and 1B), the system comprising: a bed-mountable surface or a body-mountable patch comprising a plurality of segments, wherein each of the plurality of segments comprises at least one pressure sensor, wherein the pressure sensor contacts with the pressure sensitive area of the human body.

An embodiment of the invention is directed to a method for the monitoring, prevention and management of pressure ulcers of a patient, the method comprising the steps of: collecting and profiling time-stamped whole body pressure distribution data; classifying the patient's posture; detecting and tracking the patient's limbs; assessing the risk and quality of turn of the patient; analyzing the turning schedule and utilization of caregivers; and customizing a reporting system of the patient's status.

Embodiments of the present invention provide numerous advantages over prior art techniques for monitoring and preventing pressure ulcers. Devices of the present invention are likely to be much more accurate than the manual (mostly observation-based) tests that are currently widely used in the healthcare industry, and in particular, more accurate for people with darkly pigmented skin.

Embodiments of the invention are directed to the use of algorithms for monitoring, prevention and management of pressure ulcers that include (i) time-stamped whole-body pressure distribution data collection and profiling (D-Collect module); (ii) posture classification (P-Classify module), (iii) limb detection and tracking (L-Detect module); (iv) quality of turn and risk assessment (R-Assess module); (v) turning schedule and nursing staff utilization for pressure ulcer management (T-Schedule module); and (vi) patient status reporting system customized for caregivers (S-Report module).

These and other benefits of the present invention will be apparent from the description below.

### BRIEF DESCRIPTION OF THE DRAWINGS

FIGS. 1A and 1B represent areas of the body where pressure ulcers commonly develop;

FIG. 2 represents a schematic representation of the process of monitoring and preventing pressure ulcers in accordance with an embodiment of the invention;

FIG. 3 represents the posture detection process in accordance with an embodiment of the invention;

FIG. 4 represents the sequence of steps in a processing unit in accordance with an embodiment of the invention;

FIG. 5 represents the sequence of steps in a normalization unit in accordance with an embodiment of the invention;

FIG. 6 represents the sequence of steps in a limb detection unit in accordance with an embodiment of the invention;

FIGS. 7A and 7B represent an articulated human body model in a foetus/yearner position (A) and supine position (B);

FIG. 8 represents a posture scheduling graph in accordance with an embodiment of the invention;

FIG. 9 represents a pressure-time injury (stress-recovery) model in accordance with an embodiment of the invention;

FIGS. 10A and 10B represent a real time posture classification with limb annotation in a supine position (A) and left yearner position (B) in accordance with an embodiment of the invention;

FIG. 11 represents a stress assessment graph for five at-risk regions in a test subject for various scenarios;

FIGS. 12A and 12B represent raw data derived from the S-report module in accordance with an embodiment of the invention;

FIGS. 13A and 13B represent data provided by the caregiver i.e., a Braden chart (A) and blood pressure data (B);

FIGS. 14A, 14B and 14C represent processed forms of the data derived from the S-report module, i.e. posture and limbs identified (A), posture history constructed (B), patient mobility measured (C); and

FIGS. 15A and 15B represent the risk assessment graph (A) and turning schedule (B) respectively derived from the processed data.

### DETAILED DESCRIPTION OF EXEMPLARY EMBODIMENTS

This invention relates generally to sensing and monitoring one or more conditions related to the health of the human body, such as the development, prevention and treatment of pressure ulcers. The present invention more particularly relates to a system and method of monitoring one or more pressure sensitive areas of a human body. Although aspects of the present invention have application with regard to other human body conditions, the invention will be specifically described in the context of pressure that contributes to the development of external skin ulcers (e.g., decubitus ulcers). In this context, the invention also relates to computer-implemented pressure ulcer monitoring, prevention and management methods.

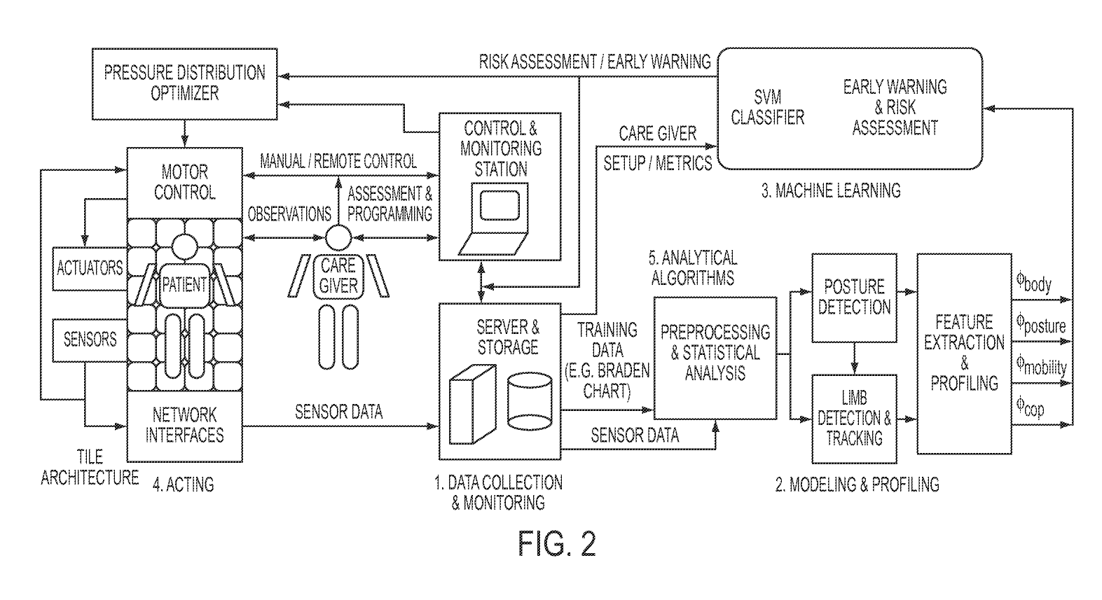

Since interaction with the contact surface such as a seat or a bed is a key cause of pressure ulcers, a smart bed or seat can provide a first line of defense in preventing them. With this goal in mind, the hospital bed can be viewed as a source of biosignal data collection, because it is where patients spend a large amount of their time. An embodiment of the invention is directed to methods of enhancing the capabilities of a hospital bed with respect to its intellectual and physical characteristics, such that it can provide cognitive support to hospital staff. More specifically, the combination of a sensor network, machine intelligence, a morphable, tiled surface equipped with programmable mechanical parts, and computer control can produce a smart bed capable of providing support to the staff that significantly improves the care, epidemiological analysis and prevention of pressure ulcers. The smart bed reduces the staff needed to turn patients. That means the nurse can spend more direct care time at the bedside of a patient assessing for complications or adverse events instead of looking for help to turn the patient. There are five aspects related to a pressure-ulcer aware smart bed system as set forth in FIG. 2.

### A. Data Collection & Monitoring

In an embodiment of the invention, in order to measure pressure over the entire body, pressure sensors are distributed over the bed's surface in an array format. Resistive and capacitive sensors are the two main types of commercially available surface pressure sensors. The technology behind large area sensing using a sensor matrix has advanced in the past decade and currently several pressure sensor arrays are offered in the market.

Although these pressure sensing devices are necessary, they are not sufficient. Off-the shelf sensor arrays can be used to capture the pressure map, but the desired level of accuracy, usefulness and sophistication of the processing required for the smart bed application, is not commercially available. Specifically, a time-stamped pressure distribution image and database that can be constructed to facilitate body part identification, posture detection/classification and body movement analysis. To simplify the monitoring, an embodiment of the invention is directed to the design of a GUI that facilitates access and visualization of relevant data.

### B. Modeling & Profiling

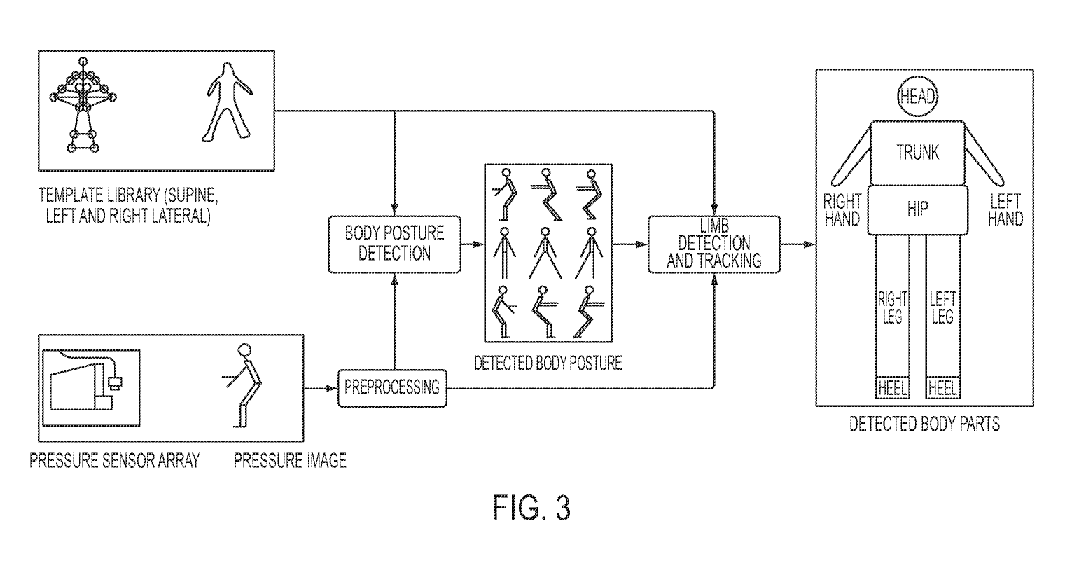

Body Posture Detection: FIG. 3 depicts an overview of the posture classification algorithm developed for the platform of the present invention. The posture detection algorithm has two main steps, i.e. training and test. The goal in training phase is to generate required data set for classification.

In an embodiment of the invention, to build the training set, a complete set of pressure maps in different postures, including but not limited to supine, left yearner, right yearner, left foetus and right foetus, are collected using the platform of the present invention. The training set goes into preprocessing unit which extracts the body segment and enhances quality of pressure images. Dimension of data is reduced by projecting images from a correlated high dimension input space into an uncorrelated low dimension data space using Principal Component Analysis (PCA). During the test, each new pressure image is projected into new dimension space. Distance between extracted features for new pressure map and the training set is measured in kNN classifier to assign labels.

Limb Detection & Tracking: Most of pressure ulcers form over bony areas of the body such as sacrum, over the hip bones, back of the head and shoulder. Limb detection allows us to track at-risk regions of the body and assess the risk associated with those parts more accurately.

In an embodiment of the invention, after classifying the patient's posture on the bed, a model is fit to the classified pressure map using an articulated human body model. A flexible and parametric human body model is developed for all postures. During the training phase, a database of human body model is generated. During testing, the most similar sample of classification algorithm serves as the initial estimation of the model parameters. After initial estimation of location, size and angle of the assigned model, blob analysis is done to tune model parameters.

Feature Extraction: It is desirable to extract relevant information from the data collection unit that can be used in the machine learning unit. There are two types of sensor data: (1) posture-independent (e.g. physiological) data such as blood pressure, and (2) posture-dependent values such as pressure, temperature or moisture on each point of body in contact with bed. Posture-dependent values will be obviously limb dependent too. For uniformity, it is assumed that each metric is sampled periodically but the sampling period for each may be different. It is assumed that there are M posture-independent, N posture-dependent metrics and L limbs to monitor. The temporal resolution of the posture-independent metrics is bounded by the sampling frequency of the data collection system. The spatial resolution of the posture-dependent metrics is confined by the distance between every two adjacent sensor nodes in the array of sensors. The concept of moments (m) is used to uniformly extract features. The first four moments are mean, variance, skewness (a measure of the asymmetry) and kurtosis (a measure of the peakedness).

The development of bed sores is directly influenced by the time duration the patient stays in each posture and how the whole body is exposed to the risk factors. For a given period of time, Δt, a vector of the first to the fourth moment or variation of the moment for both posture-dependent and independent data metrics is constructed.

### C. Machine Learning

In an embodiment of the invention, the primary goal of the unit is to apply machine learning techniques to train a model for assessing a patient's risk of developing pressure ulcer, by combining the features extracted in the modeling and profiling unit. The predictions made by this model enables the unit to (1) issue an early warning (alert) flag indicating the existence of high risk of developing ulcer and (2) control command/data for pressure redistribution around high-risk limbs (i.e. provided to the care giver or the actuation unit).

To train this risk assessor, a learning algorithm (for example support vector machine or SVM) that achieved state-of-the-art results on a variety of tasks, both within and outside the health-care domain was employed. In the training set used in an embodiment of the invention, each instance corresponds to the data collected from a particular body part of a patient at a particular time step, and is represented as a vector composed of the features discussed in the previous subsection. The label of an instance, which is manually assigned by a health-care professional, can be either a simple binary classification (i.e. whether the patient is at high risk of developing PU or not) or one of the three classes (e.g. high, moderate and low risk). Given this training set, a learning algorithm such as SVM can be used in combination with a variety of kernels to assign one of the risk levels to a test instance (if the label is one of its three classes) or a classifier for determining whether a patient has a high risk of developing ulcer (if the class label is binary). It could be argued that the binary decision returned by a classifier is not particularly useful in practice, since what is typically desired is a real value that indicates the risk of developing ulcer. In fact, this real value can be easily derived from a machine learning based classifier. Hence, a risk function R is derived that computes the risk associated with a test instance based on its distance from the hyperplane in the learning algorithm, assigning the highest (lowest) risk value to the instance that is farthest away from the hyperplane in the positive (negative) region.

### D. Acting

The general requirements on the design of the bed hardware are to provide key information for moving and manipulating the patient such that the pressure redistribution lowers the stress to the body parts that are sensitive to the pressure. The idea is to produce relevant information based on the history of stress-recovery of patient's body such that a bed with a tiled architecture and programmable mechanical parts can be programmed to react automatically or semi-automatically.

In an embodiment of the invention, the bed uses the analyzed information to perform soft, non-grasp manipulation for this purpose. The non-grasp approach is used because it is safe in that there is no attempt to grasp or constrain the patient's body. The “soft” aspect allows for fine control of the contact/pressure forces along the patient's skin. Manipulation is the key issue because manipulating/moving the patient is the most effective current practice used by nurses to prevent pressure ulcers, referred to as “turning the patient”. If the bed is equipped with a tiled architecture and programmable mechanical parts, the analyzed information would guide it how to respond automatically with no or minimal human intervention. Similarly, for semi-automatic case, a caregiver, reviews the analyzed data, makes and logs in a decision which will be followed by the bed until satisfactory re-analyzed data is observed.

### E. Analytical Algorithms

Embodiments of the invention are directed to the use of algorithms for monitoring, prevention and management of pressure ulcers that include (i) time-stamped whole-body pressure distribution data collection and profiling; (ii) posture classification, (iii) limb detection and tracking; (iv) quality of turn and risk assessment; (v) turning schedule and nursing staff utilization for pressure ulcer management and (vi) patient status reporting system customized for caregivers. In what follows, we explain each one of these algorithms.

While a combination of two or more of the algorithms may be used in an embodiment of the invention, other embodiments of the invention use a combination of three, four, five or six algorithms for the monitoring, prevention and management of pressure ulcers.

### (i) Time-Stamped Whole-Body Pressure Distribution Data Collection and Profiling (D-Collect Module)

In an embodiment of the invention, a Force Sensing Array (FSA) is used to collect pressure data on the bed. The FSA system can be (a) a flexible mat that contains a pressure sensor array (for example 32×64) uniformly distributed sensors which cover the total contact area between the subject and the bed or (b) a combination of pressure sensor patches mountable on different body limbs. The FSA system can measure interface pressure that is typical for human body, for example between 0 to 100 mmHg per sensor. The sensor mat or patch is light, thin and flexible. The electronic interface samples the sensor mat or patch in a fraction of second (e.g. 0.25 second) or more. Sensor values are time-stamped and stored as a gray scale pressure image in a database and this image is passed to a data processing unit.

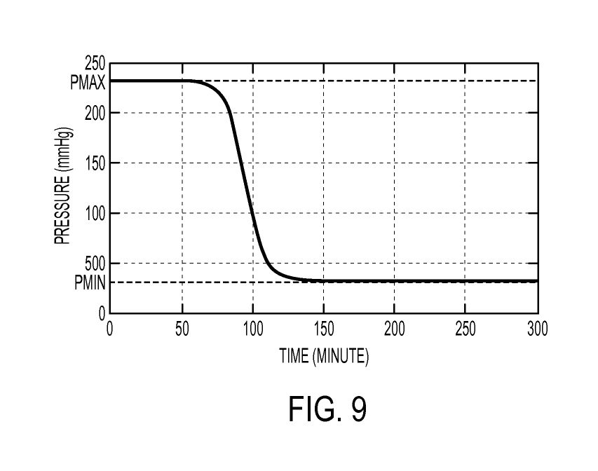

An embodiment of the design is directed to a method of generating a profile from the initial, fused sensor data in order to capture the most important metrics such as the pressure map, mobility/activity and body/limbs structure. The most critical information for a patient likely to experience a pressure ulcer can be collected directly from the sensor readings or through data fusion such as deriving probabilistic models and/or Bayesian data fusion methods. Fused data and features extracted using machine learning methods are more informative in terms of interpretation. The key profiling is based on the tissue stress-recovery which itself depends on the effects of prolonged oxygen deprivation and waste buildup. This happens when the capillary network in a region collapses, which happens when pressure is over a certain threshold Pₘₐₓ. When pressure is less than Pₘᵢₙ, tissue starts to recover. One example is shown in FIG. 9. This model is based on the region pressure which is exerted on every region of interest for a time interval using the pressure data reported by all sensors covering that particular region. Then, a worst case scenario, the maximum average pressure, is used to formulate the pressure-time cell injury (stress-recovery) threshold.

### (ii) Posture Classification (P-Classify Module)

In an embodiment of the invention, a high accuracy image-based algorithm is developed which can be used with different pressure mapping systems. The algorithm fills the gap between previous research work and a commercial product for pressure ulcer management in the sense that it can ultimately be used in bed with automatic pressure redistribution capabilities. A new algorithm is developed for classification of patient's posture on bed. An image-based preprocessing unit processes input pressure map and prepares it for classification using Principal Component Analysis (PCA) and Independent Component Analysis (ICA).

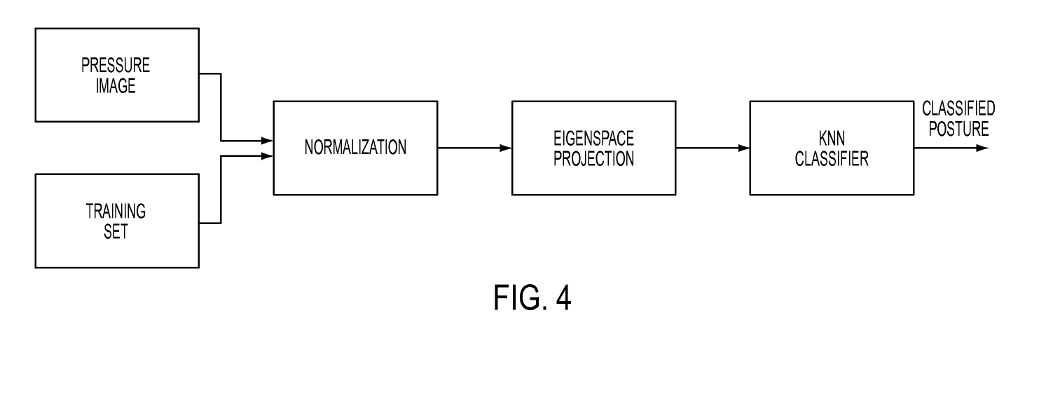

The processing unit has three main steps: Normalization, Eigenspace Projection and K Nearest Neighbor (kNN) Classifier. The normalization stage extracts the silhouette of the patient in the bed using binary image processing and prepares the training and input image with a fixed size for further processing. FIG. 4 depicts an overview of the processing unit.

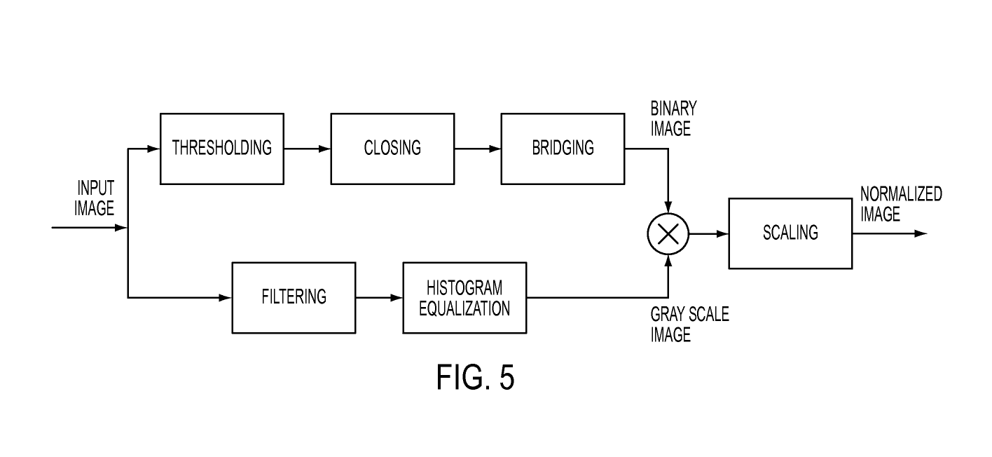

FIG. 5 represents the internal steps of this stage where the upper path generates a binary image using thresholding. In order to improve the quality of the binary image, closing and bridging morphological operations are applied on it. The resultant binary image is multiplied with filtered and equalized gray scale image and the body segment of image is scaled to a fixed size for Eigenspace projection. Smoothing is applied on gray scale pressure image using a rotationally symmetric Gaussian lowpass filter of appropriate size and deviation (for example of size 10 with standard deviation 0.5).

Projecting images into eigenspace is a procedure that could be used for appearance-based recognition algorithms. A pressure image is represented as a vector of pixels where the value of each entry is the pressure value of the corresponding sensor point. The pressure image is a point in an N-dimensional space, where N is the number of sensor points (for example 2048). In this technique, a subspace on which to project the pressure images is first selected. After subspace selection, all training images are projected into this subspace. Training set includes pressure images of different postures and different subjects considering variations within and between classes. Then, each new test image is projected into the same subspace. Projected test image is compared to all the training images by a kNN algorithm and the training images that are closest to the test image are used to identify class of test image.

In PCA model, since covariance matrix is used, second order statistics of data are captured which only have amplitude spectrum of pressure images. The phase spectrum which contains the structural information is hidden in higher order statistics. Unlike PCA which uses a Gaussian source model, the essential assumption in ICA is that the combining coefficient are non-Gaussian and mutually independent random variables allowing higher order statistics in ICA. So, the optimization in PCA is the minimization of reconstruction error from the reduced dimension data, while the optimization of ICA is the minimization of statistical dependence between the basis images. Similar to the PCA, the projection information is extracted during training phase and training database is created after projection. Cosine similarity measure is used to classify test postures in kNN.

(iii) Limb Detection and Tracking (L-Detect Module)

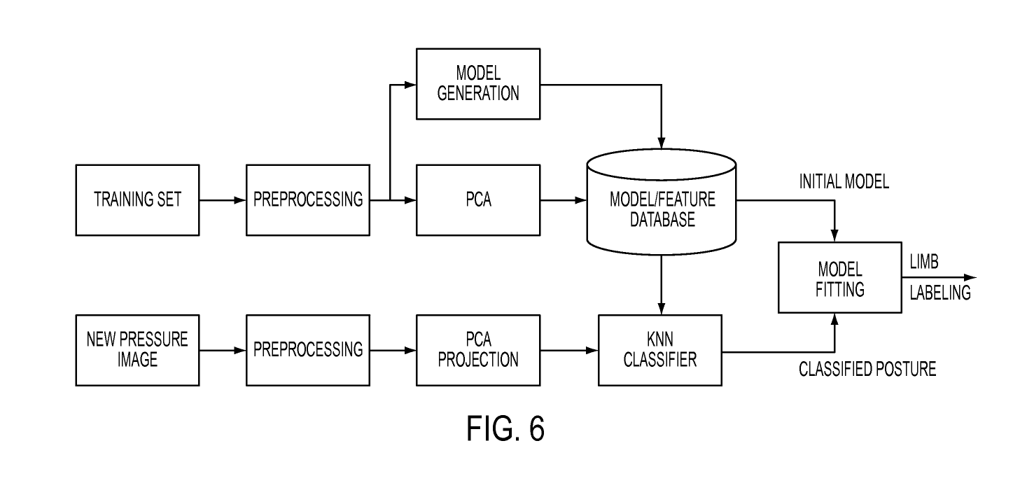

An overview of hierarchical limb detection technique is depicted in FIG. 6. In this approach, a patient's posture is first classified on the bed into different postures, for example supine, left yearner, right yearner, left foetus and right foetus. Then, a model is fit into a classified posture using an articulated human body model. The algorithm has two main steps which are a training step and a test step. The upper path in FIG. 6 is the training phase and the lower path is the test phase. The goal in the training phase is to generate required data set for both posture classification and limb detection in each posture. To do so, a database of projected training samples into lower dimension space and also a database of human model in different postures are prepared during training.

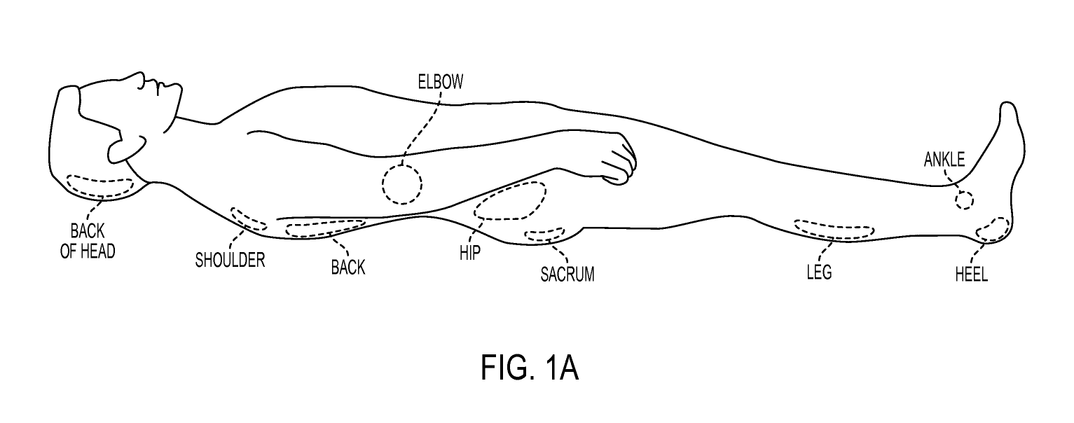

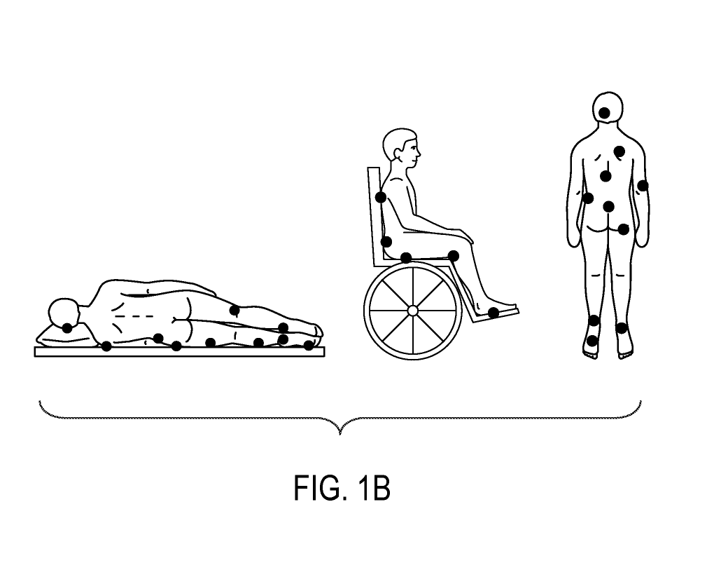

Most pressure ulcers form over bony areas of the body such as sacrum, over the hip bones, heels, back of the head, heels and shoulder. Limb detection allows us to track at-risk regions of the body and assess those parts more accurately with associated pressure statistics. FIG. 1 shows some of the high risk areas of body in different postures.

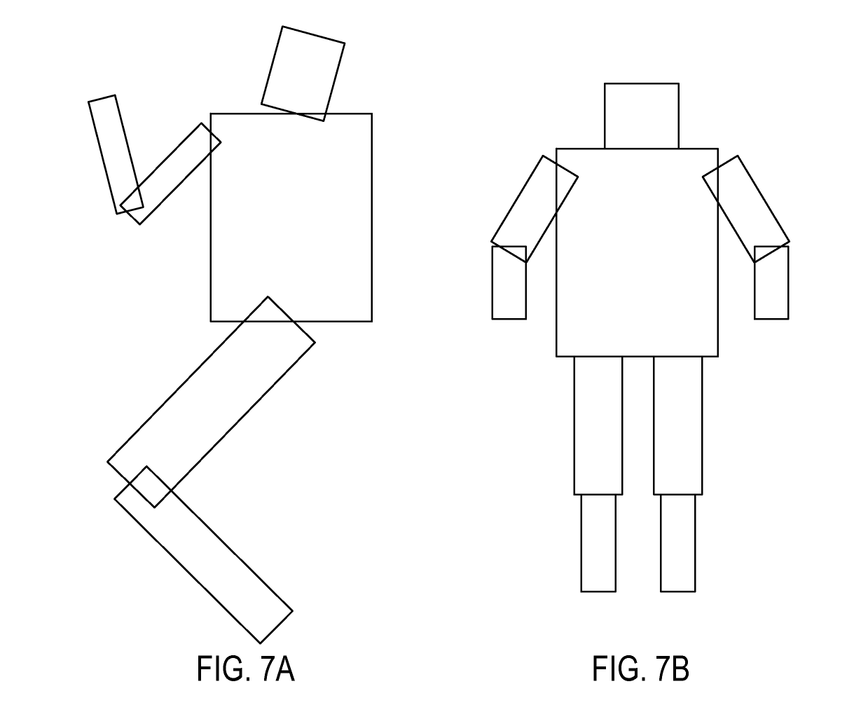

Two different articulated human body models is developed for body limb detection which are shown in FIG. 7. FIG. 7 represents a parametric model with several sizing parameters, patches and angles for head, hands and legs. FIG. 7A is used to segment body limb in Foetus and yearner postures and FIG. 7B is used to segment body limbs in supine posture. During the training phase, a database of human body is generated. During test, INN classifier chooses the most similar sample in database and associated body model of this sample is chosen to be an initial estimation of the model parameters for new test map. The algorithm fits head and back area, legs and hand patches respectively by doing a hierarchical search around the initial parameters.

The algorithm detects body limbs through a sequence of data processing:
1. It imports Binary pressure image with associated current posture;
2. It extracts the skeleton using triangulation technique;
3. It prunes the skeleton tree; and
4. It segments the body parts using resultant tree and associated posture

Constraint triangulation technique is used to divide the body into triangular meshes. A tree is generated by connecting the centroid of all triangles and it is pruned to achieve the skeleton of the pressure image. Then, the pruned tree is decomposed into different subsegments, where each subsegment can be considered as a limb.

### (iv) Quality of Turn and Risk Assessment (R-Assess Module)

Based on a stress-recovery model which can be personalized (see FIG. 9), an embodiment of the invention is directed to the use of a mathematical formulation to measure stress. The mathematical formulas used include, but are not limited to the following relationships:
1. The maximum average pressure based on pressure sensor data:

$${P}_{i} ( x ) = {max}_{s ∈ {S}_{i} ( x )} \{ \frac{1}{Δ t} {\int }_{0}^{Δ t} {p}_{s} ( t ) d t \}$$

In the above formula, ΔT denotes the time interval, S₁(x) is the set of all sensors covering region i for posture x and pₛ is the sensor reading from the pressure mat.
2. The critical time of exposure:

$${T}_{inj} ( P ) = \{ \begin{matrix}∞ & P \leq {P}_{min} \\ 0 & P > {P}_{max} \\ {T}_{0} + \frac{1}{λ} ln ( \frac{{P}_{max} - {P}_{min}}{P - {P}_{min}} - 1 ) & otherwise\end{matrix}$$

where P is a specific amount of pressure, λ is the pressure-time injury constant and Pₘₐₓ and Pₘᵢₙ are stress and recovery thresholds, respectively.
3. The stress-recovery accumulation function:

$$Φ ( {S}_{0} , P , Δ t ) \dot{=} \{ \begin{matrix}0 & if P \leq {P}_{min} \\ {S}_{0} + \frac{Δ t}{{T}_{inj} ( P )} & otherwise\end{matrix}$$

where S₀ denotes the starting stress. There are three key properties on the stress-recovery accumulation function which are (a) non-negativity, (b) homomorphism and (c) non-recovery above threshold. These three properties make this function extremely useful in risk assessment and prediction. We have successfully integrated this accumulation function in optimization of ulcer prevention management including for optimizing nursing efforts and turning schedules.

### (v) Turning Schedule and Nursing Staff Utilization for Pressure Ulcer Management (T-Schedule Module)

The current standard for prevention is to reposition at-risk patients every two hours. But, each patient has different needs based on overall vulnerability and damaged skin areas. A fixed schedule may either result in some patients getting ulcers, or nurses being overworked by turning some patients too frequently.

An embodiment of the invention is directed to an algorithm that finds an efficient repositioning schedule for bed-bound patients based on their risk of ulcer development. The proposed algorithm uses data from a pressure mat assembled on the bed's surface or from body-mountable pressure sensor patches and provides a sequence of next positions and the time of repositioning for each patient. The patient-specific turning schedule minimizes the overall cost of nursing staff involvement in repositioning the patients while simultaneously decreasing the chance of pressure ulcer formation. The method creates a finite_{:}duration repositioning schedule that optimizes nurse efforts while preventing pressure ulcers. This is done based on the combined stress-recovery model extracted from the pressure sensor data explained earlier.

An optimal posture-changing schedule can be found using a variant of the resource-Constrained Shortest Path (CSP) problem that allows non-linear constraints. For the posture scheduling problem, every T minutes a decision is made to reposition the patient to a specific new posture or to leave him/her in the same position. A given posture schedule can be thought of as a particular path through a graph encoding all possible decisions. Each node in the path represents the posture chosen at a particular decision point, and each edge can encode the resource usage and cost.

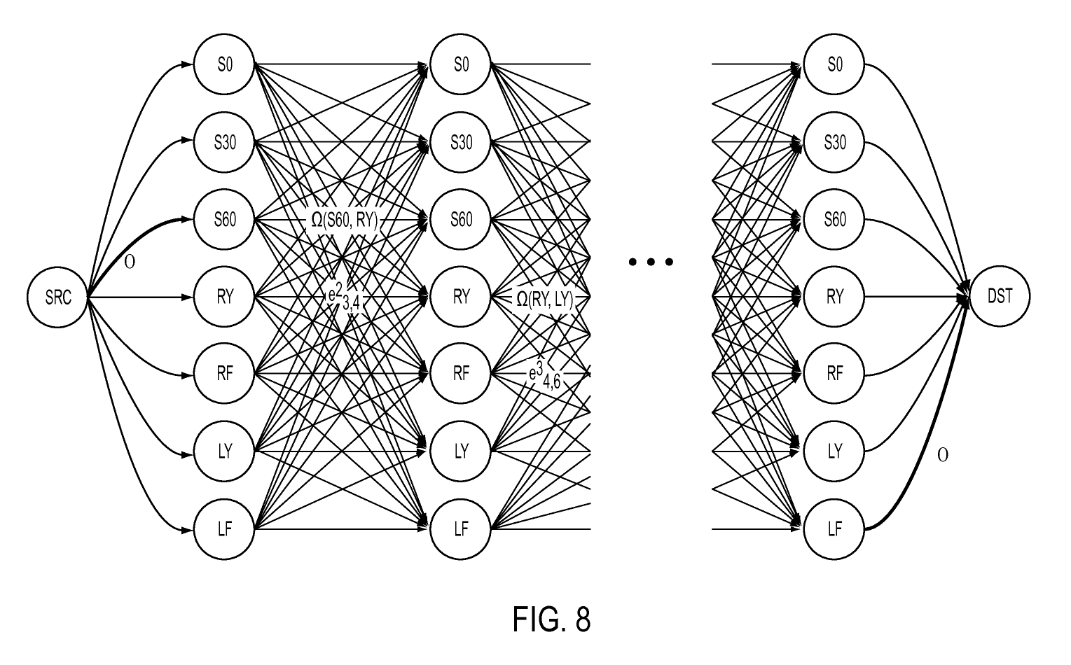

A special graph G(V,E) is built in stages, with each stage representing a particular decision point. A stage added at time t adds specific number of nodes, with each node representing a different choice of posture. In a posture schedule, exactly one posture is chosen at each time. This is facilitated by adding directed edges. The cost associated with the edge, denote as Ω(Xᵢ,Xⱼ), is the transition cost between the two postures. Finally, the source (SRC) and destination (DST) nodes are added. Every posture in the final stage is connected to the destination node at zero cost, since there is no constraint on the final posture. If the initial posture is unspecified, then the source node is connected to all postures in stage 1 at zero cost; otherwise, it is only connected to the specified initial posture. FIG. 8 shows the constructed scheduling graph for our set of postures, i.e. {Supine (S0°, S30°, S60°), Right Yearner (RY), Right Foetus (RF), Left Yearner (LY), and Left Foetus (LF)}. The highlighted path represents an example of posture schedule.

The approach for solving the CSP is to find the shortest path and see if the resource constraints are violated. If so, the second shortest path is tried. This process is repeated until a path is found that does not violate the constraints. This approach is employed to solve the scheduling problem, as it does not depend on linear resource constraints. The nth optimal schedule can be found with a k-shortest path algorithm. The lazy evaluation of k-shortest path is used to efficiently find the shortest paths.

### (v) Patient Status Reporting System Customized for Caregivers (S-Report Module)

The data collected and analyzed needs to be communicated effectively to caregivers for monitoring, prevention and management of ulcer. An embodiment of the invention is directed to the development of an efficient and user-friendly (for medical staff and caregivers) visualization and summary report of the data that is collected and processed. This embodiment includes but not limited to the following five key components:
1. Raw Data: This software module collects data from a large number (e.g. 2048) of pressure sensors which is embedded in bed or from pressure sensor patches mounted on body and stores it in a list for further processing. Data is shown in color-coded graph for recognizing spots that are under high pressure and stress. The module can also show statistics of sensors data that includes but not limited to maximum, minimum, average and variance.
2. Caregiver's Data: Using this module, caregivers can optionally enter data about the patient's status and their observations that they view critical for risk assessment. These options include but not limited to Braden Chart selection, blood pressure, bed inclination, etc.
3. Processed Data: The result of posture classification, limb detection and mobility of patient in bed (large as well as small movements) are captured, summarized and pictured for any time span of interest (from a few minutes to several hours). The summary report includes but not limited to (a) number of turns, (b) exact, average and maximum time between turns, (c) quality of turns (based on risk assessment) and (d) risk assessment and predictor for each limbs as well as for whole-body.
4. Risk Assessment: The risk assessed by R-Assess module is normalized and visualized using a two-dimensional graph for caregiver's quick comprehension of the status.
5. Resource-Turning Schedule: The system generates the best monitoring and treatment plan for the next several hours and show it like a histogram for treatment planning and wound care management.

### WORKING EXAMPLES

### (i) Time-Stamped Whole-Body Pressure Distribution Data Collection and Profiling (D-Collect Module)

When a region is exposed to a safe pressure (less than Pₘᵢₙ), the stressed tissue enters into a phase of recovery. The investigation on the off-loaded recovery interval shows that recovery time increases with load duration and load pressure. Empirical data have shown a recovery time of several minutes (for example about 15 minutes after 40 minutes of continuous heel pressure loading by measuring heel blood perfusion) by means of laser Doppler imaging. We also compared the recovery response of full relief and partial relief. The experimental results of these studies indicate the recovery time is in the order of several minutes, which is effectively instantaneous compared to standard repositioning intervals greater than 1 hour.

Based on the data collected, a cell stress-recovery model as shown in FIG. 9 can be created. This model can be personalized for each individual based on race, gender, age and health conditions. It can also be customized for various body parts with different tissue cells. For example, according to the model shown in FIG. 9, a pressure of 150 mmHg can be applied on a body region for almost 90 minutes without risk of ulcer development. But, a longer period than that would be risky and may damage tissues causing onset of ulceration.

The next experiment was a simulation study to conduct comparison between the risk factors for different body limb tissues. For each object, a different layer was used underneath the subject in terms of softness to mimic the role of various body tissues and be able to show the risk factor. Bony limbs were simulated with harder layer and muscles with softer one. The left object had the hardest layer underneath and the right one has the softest layer. The overall pressure of all three objects is the same since the weight and the contact area are the same.. However, the pressure distribution is expectedly different. Table I shows the extracted features (four moments) for these three objects. As expected, the mean values are approximately the same for all. The harder the layer, the higher the variance over the surface. To calculate the risk factor (R), a simple normalization method was applied to compute relevant weight (ωᵢ) for each feature (i.e. moments mᵢ). The last column of this table shows the risk factor (0<R<1) for each scenario. The results indicate the harder the layer or tissue, the higher the risk factor. The experiment validates that it is possible to assess the risk associated with various limbs and the whole body.

TABLE I

| Tissue | Mean | Variance | Skewness | Kurtosis | Risk |
|---|---|---|---|---|---|
| Softness | (m₁) | (m₂) | (m₃) | (m₄) | (R) |
| Hard | 0.0138 | 0.0074 | 9.4657 | 98.6307 | 0.8460 |
| Medium | 0.0135 | 0.0065 | 4.7368 | 25.4770 | 0.4995 |
| Soft | 0.0129 | 0.0016 | 3.6443 | 16.4165 | 0.2712 |

### (ii) Posture Classification (P-Classify Module)

Table II summarizes experimental results obtained using the posture detection algorithm of the present invention. Each column of the matrix represents instances in actual class while each row of this matrix represents the instances in a predicted class. For example, the entries of the first column of the matrix have the following meaning: 99.2% of actual Right Foetus instances are predicted correctly while 0.7% of actual Right Foetus instances are erroneously predicted as Right Yearner and 0.1% as Supine. The overall accuracy (correct predictions) of the method is 97.7%.

TABLE II
PCA CONFUSION MATRIX(%)

| | Right | Left | Right | Left | |
|---|---|---|---|---|---|
| | Foetus | Foetus | Yearner | Yearner | Supine |
| Right | 99.2 | 0 | 9.3 | 0 | 0.1 |
| Foetus | | | | | |
| Left | 0 | 99.6 | 0 | 0.2 | 0.1 |
| Foetus | | | | | |
| Right | 0.7 | 0 | 90.7 | 0 | 0.3 |
| Yearner | | | | | |
| Left | 0 | 0.4 | 0 | 99.8 | 0.2 |
| Yearner | | | | | |
| Supine | 0.1 | 0 | 0 | 0 | 99.3 |
| Recall | 99.2 | 99.6 | 90.7 | 99.8 | 99.3 |
| Precision | 91.3 | 99.7 | 98.9 | 99.4 | 99.9 |

| | |
|---|---|
| Accuracy | 97.7 |

(iii) Limb Detection & Tracking (L-Detect Module)

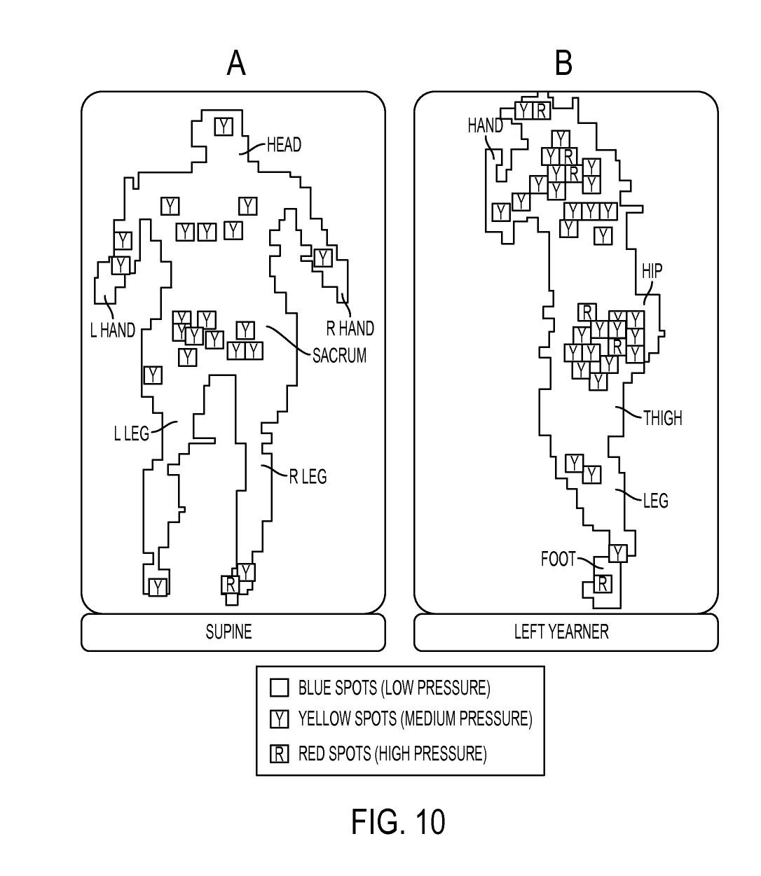

The limb detection method applied to various subjects runs very fast and identifies the main limbs real time with very high accuracy. Our system is capable of identifying the limbs and annotating the information as shown in FIG. 10. Note that three labels “blue”, “yellow” and “red” are used in FIG. 10 to indicate the general concept of various levels of pressure captured and pictured by our system. The real images are very colorful with many levels of intensity and shades indicating very accurate pressure distribution across body.

### (iv) Quality of Turn and Risk Assessment (R-Assess Module)

To verify the effectiveness of the proposed CSP optimization algorithm, we collected pressure data from a commercial pressure mat for three different subjects lying on a hospital bed. Every subject was positioned in seven different postures, {Supine (S0°, S30°,S60°), Right Yearner (RY), Right Foetus (RF), Left Yearner (LY), and Left Foetus (LF)}. The difference between the three supine postures is the angle of inclination of the bed. Stress is only tracked for a finite number of at-risk regions. These regions and the postures which induce loading pressures on these regions is tabulated in TABLE III. Not all the regions listed for a given posture are loaded in that posture for every subject.

TABLE III

| No. | At-Risk Regions | Corresponding Postures |
|---|---|---|
| 1 | back of head | {Supine} |
| 2 | right head | {Right Yearner, Right Foetus} |
| 3 | left head | {Left Yearner, Left Foetus} |
| 4 | right back | {Supine, Right Yearner, Right Foetus} |
| 5 | left back | {Supine, Left Yearner, Left Foetus} |
| 6 | right shoulder | {Right Yearner, Right Foetus} |
| 7 | left shoulder | {Left Yearner, Left Foetus} |
| 8 | right elbow | {Supine, Right Yearner, Right Foetus} |
| 9 | left elbow | {Supine, Left Yearner, Left Foetus} |
| 10 | center sacrum | {Supine} |
| 11 | right buttock | {Supine, Right Yearner, Right Foetus} |
| 12 | left buttock | {Supine, Left Yearner, Left Foetus} |
| 13 | right hip | {Right Yearner, Right Foetus} |
| 14 | left hip | {Left Yearner, Left Foetus} |
| 15 | right leg | {Supine, Right Yearner, Right Foetus} |
| 16 | left leg | {Supine, Left Yearner, Left Foetus} |
| 17 | right heel | {Supine} |
| 18 | left heel | {Supine} |
| 19 | right ankle | {Right Yearner, Right Foetus} |
| 20 | left ankle | {Left Yearner, Left Foetus} |

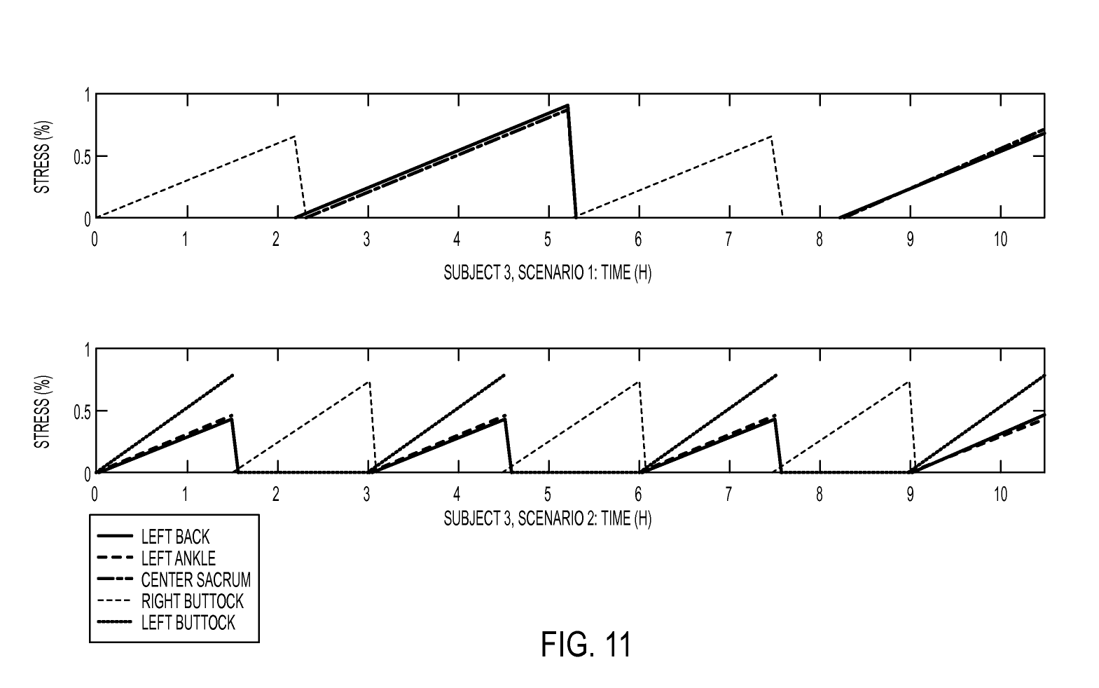

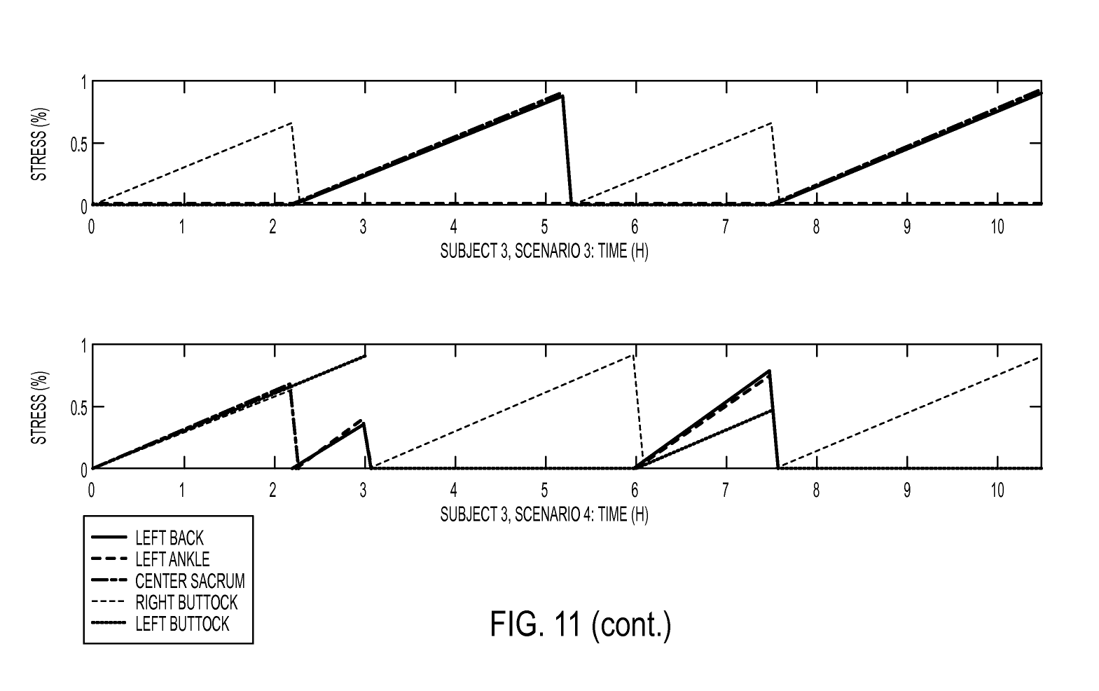

FIG. 11 shows the stress accumulation for five at-risk regions that may have red spots in different scenarios for a test subject. The stress was normalized into 0.0 to 1.0 interval for every region i. For Sc1 scenario, FIG. 11 shows that the stress reaching to the threshold in left and right regions is the main reason for repositioning from one side to another side. For Sc3, the figure shows that stress in left and right buttocks increases to the threshold in left and right sides, respectively and gets reset in the other side. One or the other buttock is an at-risk region for every posture, so scenario two (Sc2) results in a turn every hour and a half, alternating between postures loading the left buttock and those loading the right buttock. It is clear from FIG. 11 that alternating between left and right postures in the subject, puts no pressure on sacrum area and its stress stays zero through the entire schedule. In scenario four (Sc4), since both red spots happened to be in the left side, no subject can stay in the left side postures longer than one hour and a half. The figure for Sc4 also shows in subject #3, since the left buttock is exposed to pressure more than Pₘᵢₙ for both S0° and left foetus, stress keeps increasing in left buttock in the first 3 hrs of schedule even though there is a repositioning after 2:15 hrs.

### (v) Turning Schedule and Nursing Staff Utilization for Pressure Ulcer Management (T-Schedule Module)

### Optimizing Nursing Resources

Major repositionings from side postures to the Supine position or from one side to another side often require two nurses, while the minor changes such as going from Foetus to Yearner in the same side or changing the inclination of the torso portion of the bed can be accomplished by only one nurse (for electric beds, changing inclination is accomplished by pushing a button, but many immobilized patients are still incapable of doing this by themselves). The cost is calculated using our cost function, with the results shown in TABLE IV. We consider it takes a few (for example about five) minutes for a nurse to come into the room to move the patient, so τ₀=5 min. From the table, going from right yearner to supine takes two nurses five minutes to get there and ten minutes to reposition the patient for a total of Ω(RY,S0°)=30 min.

### Optimizing Turning Schedule

Four treatment scenarios were created based on typical patient conditions. The first is for immobile, but otherwise healthy patients, and the other three are based on patients with reddened areas of skin. Reddened skin is the first symptom of ulcer formation. By reducing pressure exposure to these areas, pressure ulcers can often be prevented. The list of scenarios follows.
1. Sc1: All of the body areas are healthy without any symptom of ulceration
2. Sc2: Reddened skin on the right and left buttocks
3. Sc3: Reddened skin on the central sacrum area
4. Sc4 Reddened skin on the right ankle and left back

TABLE IV

| | S0° | S30° | S60° | RY | RF | LY | LF |
|---|---|---|---|---|---|---|---|
| S0° | 0 | τ₀ | τ₀ | 2(τ₀ + | 2(τ₀ + | 2(τ₀ + | 2(τ₀ + |
| | | | | 10) | 10) | 10) | 10) |
| S30° | τ₀ | 0 | τ₀ | 2(τ₀ + | 2(τ₀ + | 2(τ₀ + | 2(τ₀ + |
| | | | | 10) | 10) | 10) | 10) |
| S60° | τ₀ | τ₀ | 0 | 2(τ₀ + | 2(τ₀ + | 2(τ₀ + | 2(τ₀ + |
| | | | | 10) | 10) | 10) | 10) |
| RY | 2(τ₀ + | 2(τ₀ + | 2(τ₀ + | 0 | τ₀ + 5 | 2(τ₀ + | 2(τ₀ + |
| | 10) | 10) | 10) | | | 15) | 15) |
| RF | 2(τ₀ + | 2(τ₀ + | 2(τ₀ + | τ₀ + 5 | 0 | 2(τ₀ + | 2(τ₀ + |
| | 10) | 10) | 10) | | | 10) | 15) |
| LY | 2(τ₀ + | 2(τ₀ + | 2(τ₀ + | 2(τ₀ + | 2(τ₀ + | 0 | τ₀ + 5 |
| | 10) | 10) | 10) | 15) | 15) | | |
| LF | 2(τ₀ + | 2(τ₀ + | 2(τ₀ + | 2(τ₀ + | 2(τ₀ + | τ₀ + 5 | 0 |
| | 10) | 10) | 10) | 15) | 15) | | |

Studies have shown that, depending on the body structure and the physiological state, patients can tolerate a turning schedule of two to five hours before developing an ulcer. Based on this, all healthy regions are assigned a risk threshold corresponding to two to three hours of permissible exposure for an average pressure, and reddened regions are assigned a threshold corresponding to an hour and a half of exposure. In general, physicians may choose to raise or lower the threshold for every region based on overall patient risk, or lower specific higher risk regions. In this experiment, our scenarios are kept intentionally simple to better demonstrate the properties of the optimal schedule.

TABLE V shows the sequence of computed postures for all of the subjects. In the first scenario (Sc1), an obvious solution is to always move from left to right sides every three hours because there is no overlap in body regions. For the first subject, we see transitions from the left side to the supine. This occurs because for this subject, the patient is not placing excessive pressure on the left buttocks in the supine position. The third scenario (Sc3) is similar, except the sacrum has a red-spot, forcing no subject to choose supine.

TABLE V

| | Time Duration | | | | | | | | | | | | | | | |
|---|---|---|---|---|---|---|---|---|---|---|---|---|---|---|---|---|
| Trial | 0:45 | 1:30 | 2:15 | 3:00 | 3:45 | 4:30 | 5:15 | 6:00 | 6:45 | 7:30 | 8:15 | 9:00 | 9:45 | 10:30 | No. | C(Q) |

Subject #1

| | | | | | | |
|---|---|---|---|---|---|---|
| Sc1 | RF | LY | RY | LY | 320 | 120 |

| | | | | | | | | | |
|---|---|---|---|---|---|---|---|---|---|
| Sc2 | RY | LY | RY | LF | RF | LY | RY | 256 | 240 |

| | | | | | | |
|---|---|---|---|---|---|---|
| Sc3 | RY | LF | RF | LF | 320 | 120 |

| | | | | | | | |
|---|---|---|---|---|---|---|---|
| Sc4 | RY | LY | RF | LY | S60° | 192 | 150 |

Subject #2

| | | | | | | |
|---|---|---|---|---|---|---|
| Sc1 | LY | S60° | LF | S60° | 720 | 90 |

| | | | | | | | | | |
|---|---|---|---|---|---|---|---|---|---|
| Sc2 | LY | S60° | LF | S30° | LF | S60° | LF | 1080 | 180 |

| Sc3 | RY | LF | | S60° | LY | 48 | 100 |
|---|---|---|---|---|---|---|---|
| Sc4 | S0° | LF | S30° | LF | S60° | 1476 | 120 |

Subject #3

| | | | | | | |
|---|---|---|---|---|---|---|
| Sc1 | RF | LY | RY | LF | 320 | 120 |

| | | | | | | | | | |
|---|---|---|---|---|---|---|---|---|---|
| Sc2 | LF | RY | LF | RY | LY | RF | LY | 256 | 240 |

| | | | | | | |
|---|---|---|---|---|---|---|
| Sc3 | RY | LF | RY | LY | 320 | 120 |

| | | | | | | | |
|---|---|---|---|---|---|---|---|
| Sc4 | S0° | LF | RF | LY | RY | 64 | 150 |

One or the other buttock is an at-risk region for every posture, so scenario two (Sc2) results in a turn every hour and a half, alternating between postures loading the left buttock and those loading the right buttock. In scenario four (Sc4), it was possible for the first subject to completely avoid postures with the left back and right ankle, resulting in a very low-cost schedule. The other subjects had to be repositioned every one hour and half. The greater the frequency of repositioning the greater the cost of nursing. The last column in TABLE V shows C(Q), the total nursing effort for T=10.5-hour schedule. The column next to last shown as “No.” indicates the number of optimal solutions for each scenario. In order to choose one of these optimal solutions, our algorithm takes the patient's comfort and/or restrictions into account. For example, many patients prefer supine for ease of in-bed activities such as eating, watching TV and talking to visitors. An example of patient's restrictions is those with feeding tubes that cannot be put in side positions.

### (vi) Patient Status Reporting System Customized for Caregivers (S-Report Module)

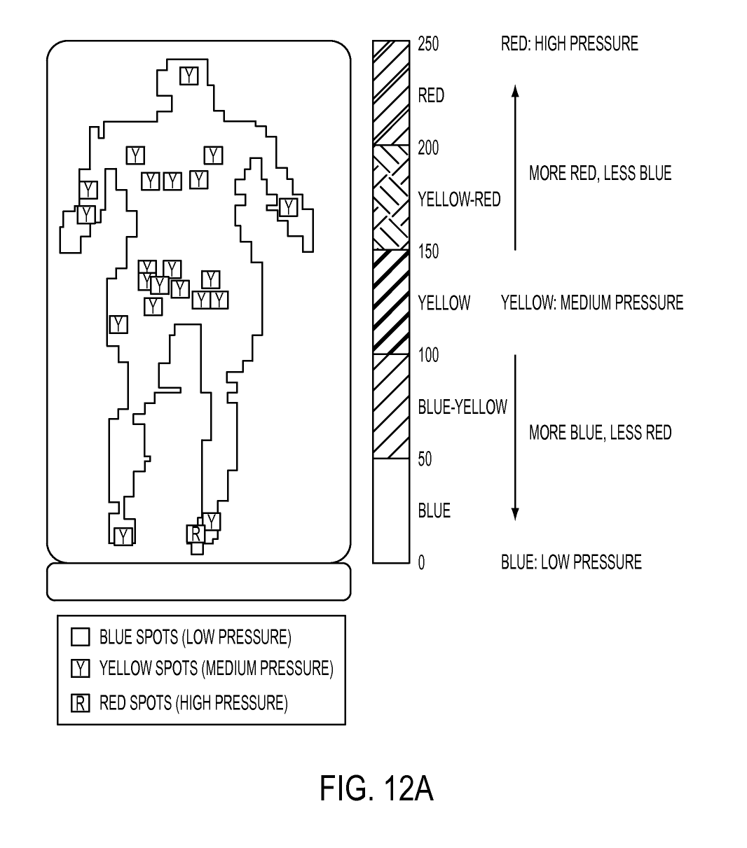

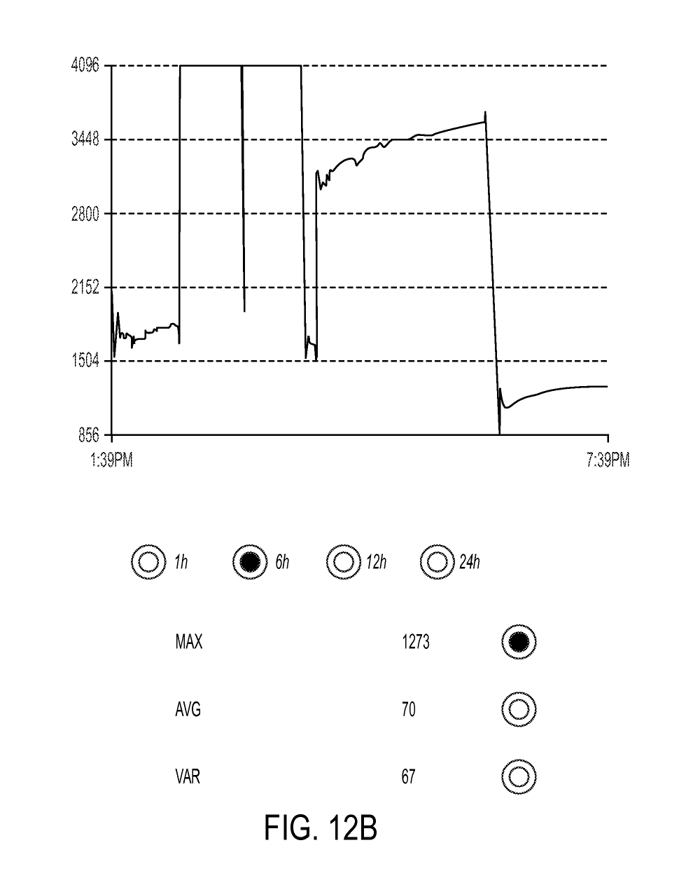

The S-Report module includes five major components. Each of these five components that visualize data and produce reports are set forth in FIGS. 12A, 12B, 13A, 13B, 14A, 14B, 14C, 15A and 15B.

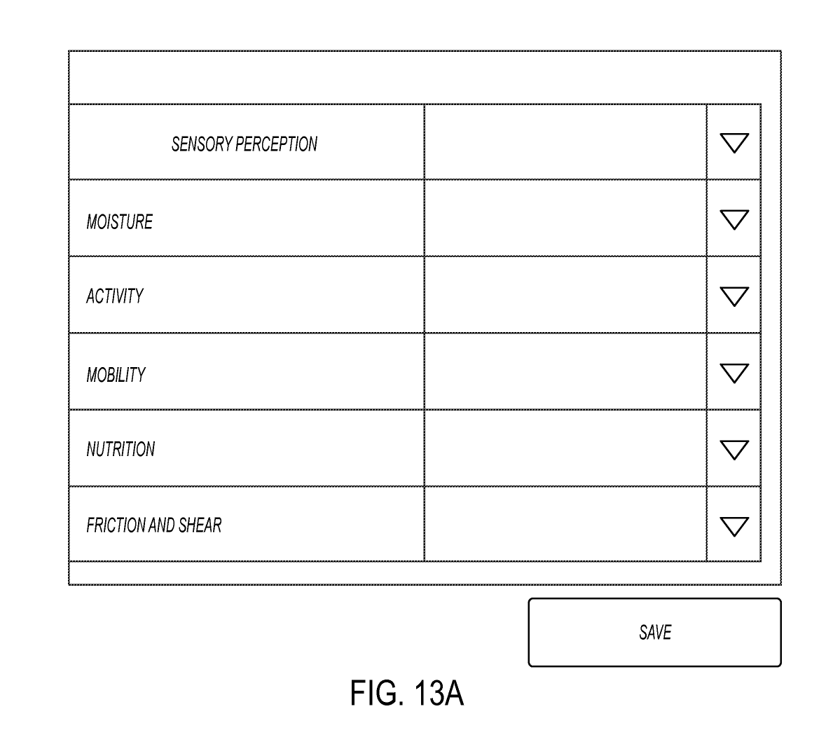

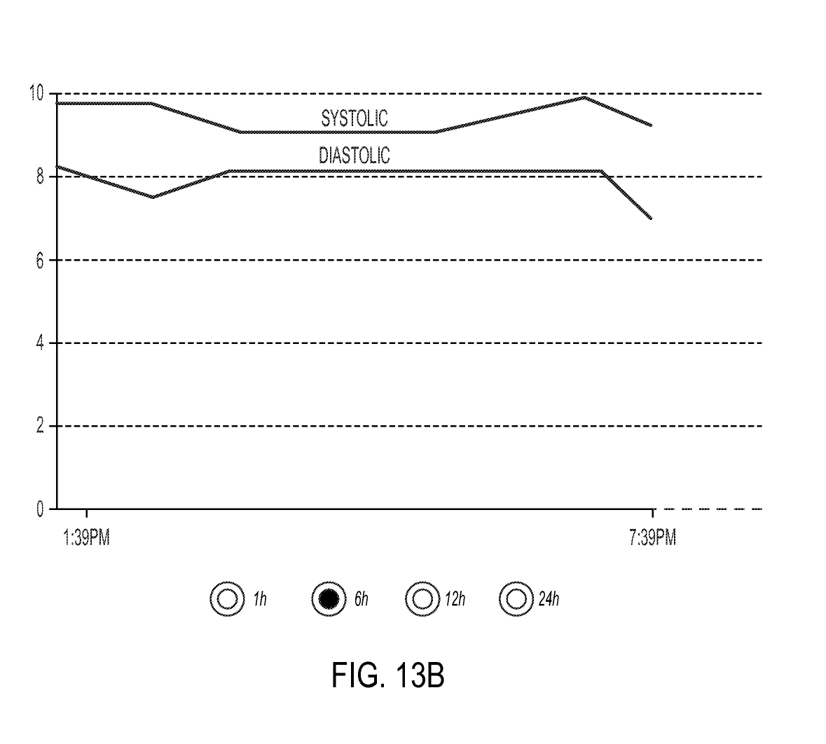

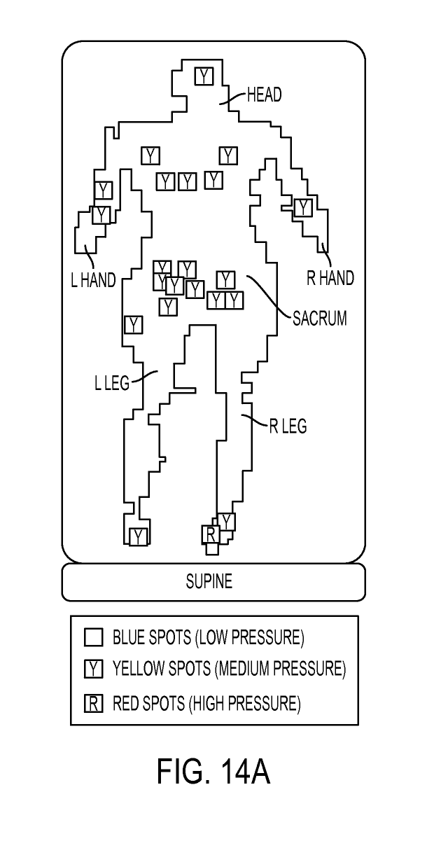

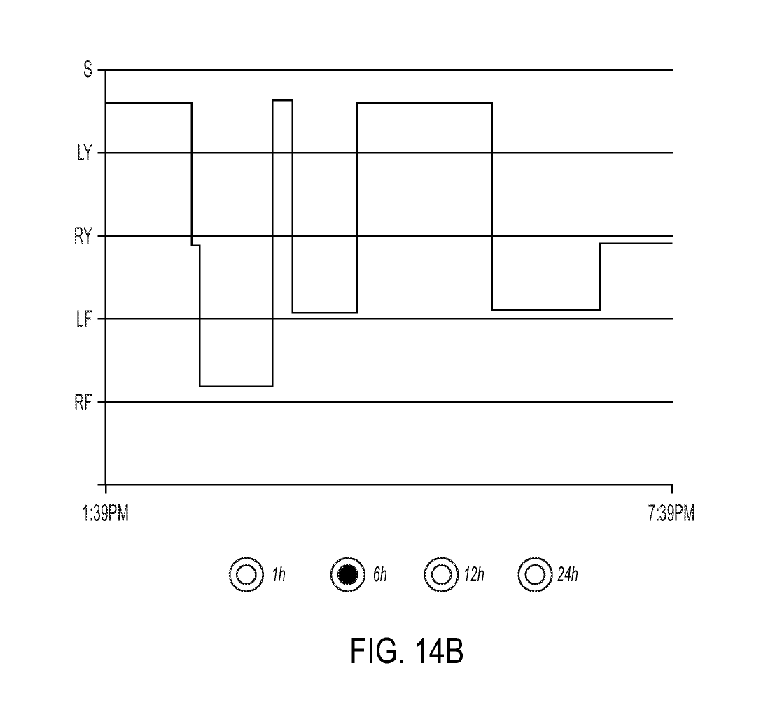

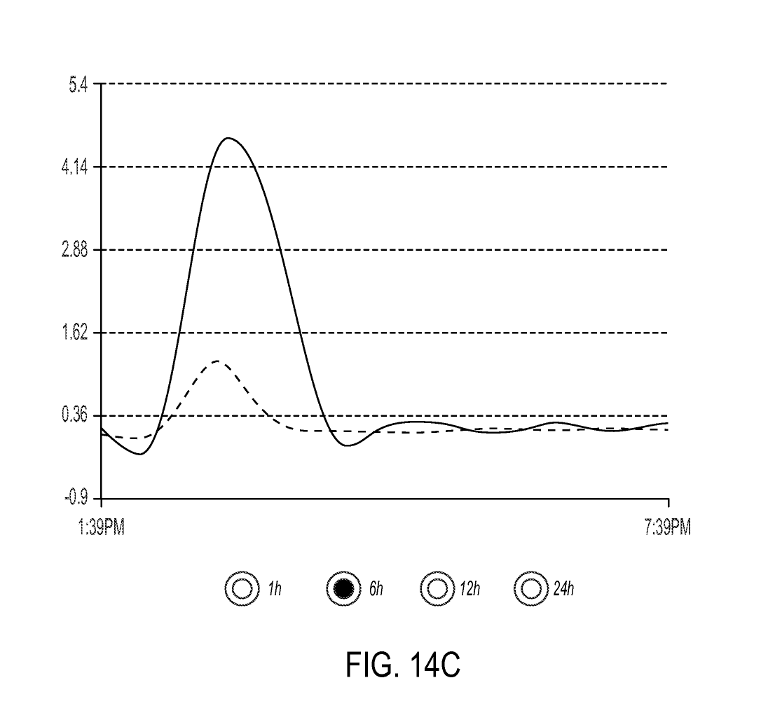

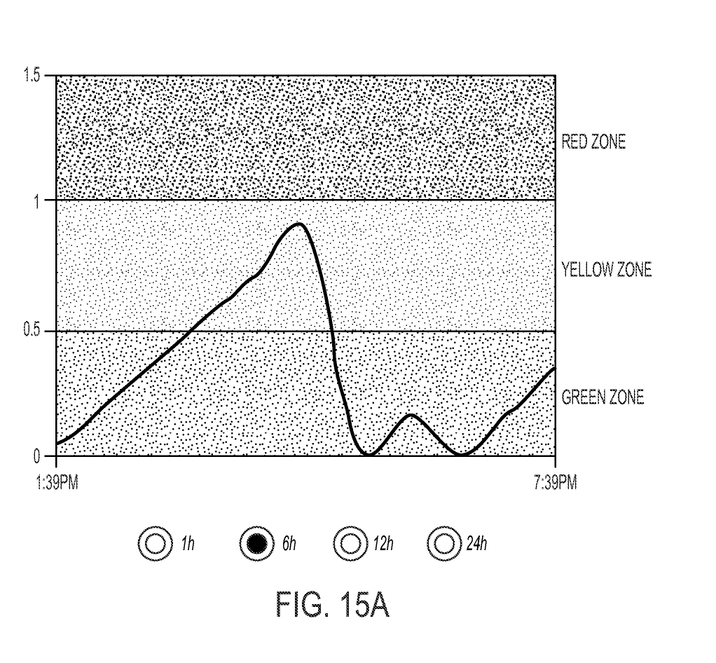

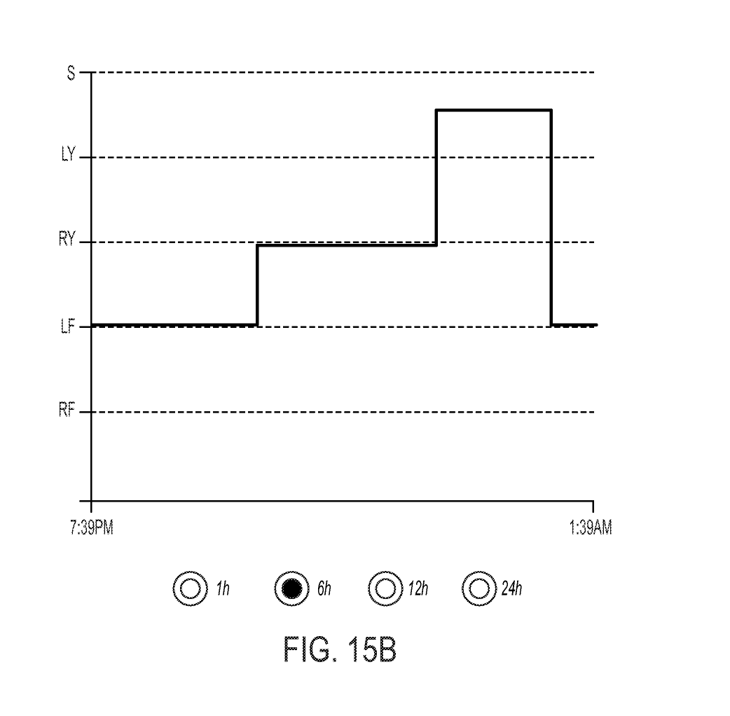

FIGS. 12A and 12B represent raw data derived from the S-report module. FIGS. 13A and 13B represent data provided by the caregiver i.e., a Braden chart (A) and blood pressure data (B). FIGS. 14A, 14B and 14C represent processed forms of the data derived from the S-report module, i.e. posture and limbs identified (A), posture history constructed (B), patient mobility measured (C). Note that in FIGS. 12A and 14A three labels “blue”, “yellow” and “red” are used to indicate the general concept of various levels of pressure captured and pictured by our system. The real images are very colorful with many levels of intensity and shades indicating very accurate pressure distribution across body. FIG. 14C shows two types of mobilities, i.e. large movements (curve with solid line) and small displacements (curve with broken line). FIGS. 15A and 15B represent the risk assessment graph (A) and turning schedule (B) respectively derived from the processed data. FIG. 15A is a visualization of risk level, for the whole body as well as each limb. If the risk is low, the curve stays in the green (low) zone. Once the risk starts to increase, the curve enters the yellow (middle) zone. And if the stress is not removed, it eventually enters the red (top) zone. This visualization can be used for real time monitoring and/or risk prediction. The turning schedule, as shown in FIG. 15B, is automatically computed and recommended, based on patient's history and physiological data, to minimize the risk of developing pressure ulcer.

While various embodiments have been described herein, it should be apparent that various modifications, alterations, and adaptations to those embodiments may occur to persons skilled in the art with attainment of at least some of the advantages. For example, different materials, analysis/optimization parameters and constants, may be used than those described above for certain components. The disclosed embodiments are therefore intended to include all such modifications, alterations, and adaptations without departing from the scope of the embodiments as set forth herein. Thus, the present invention is well adapted to carry out the objects and attain the ends and advantages mentioned above as well as those inherent therein. While preferred embodiments of the invention have been described for the purpose of this disclosure, changes in the construction and arrangement of parts and the performance of steps can be made by those skilled in the art, which changes are encompassed within the spirit of this invention as defined by the appended claims.

What is claimed is:

1. A method of monitoring pressure applied to a pressure sensitive area of a human body, the method comprising:
contacting a plurality of pressure sensors with pressure sensitive areas of the human body;
determining body posture;
tracking and determining the position of limbs of the human body;
collecting data from the pressure sensors mounted on a bed's surface such as a pressure sensor mat;
extracting features from the collected data;
training a model by using the extracted features; and
determining the level of risk associated with the development of a pressure ulcer.

2. The method according to claim 1, wherein the pressure sensors are mounted on a bed's surface.

3. The method according to claim 1, wherein the pressure sensors are mounted on a mat that is placed on a bed's surface.

4. The method according to claim 1, wherein the pressure sensors are mounted on the human body.

5. The method according to claim 4, wherein the pressure sensors are in the form of pressure sensor patches.

6. The method of claim 2, wherein the plurality of pressure sensors are distributed over the bed's surface.

7. The method of claim 1, wherein the pressure sensors provide a pressure distribution image.

8. The method of claim 1, wherein the collected data provides body posture detection, limb detection and tracking, posture independent sensor data and posture dependent sensor data.

9. A method for the monitoring, prevention and management of pressure ulcers of a patient, the method comprising the steps of:
collecting and profiling time-stamped whole body pressure distribution data;
classifying the patient's posture;
detecting and tracking the patient's limbs;
assessing the risk and quality of turn of the patient;
analyzing the turning schedule and utilization of caregivers; and
customizing a reporting system of the patient's status.

10. A method for the monitoring, prevention and management of pressure ulcers of a patient, the method comprising the step of:
collecting and profiling time-stamped whole body pressure distribution data, wherein the profiling step is based on tissue stress recovery as a function of pressure and time.

11. The method of claim 10 further comprising the step of:
classifying the patient's posture using pressure input data, wherein the classifying step extracts a silhouette of the patient using binary image processing.

12. The method of claim 11 further comprising the step of:
detecting and tracking the patient's limbs through a sequence of data processing including the steps of importing pressure image associated with the patient's current posture, extracting a skeleton tree from the image using triangulation technique, pruning the skeleton tree and segmenting the patient's limbs using the skeleton tree and associated posture.

13. The method of claim 12 further comprising the step of:
assessing the risk and quality of turn of the patient using a stress recovery model.

14. The method of claim 13 further comprising the step of:
analyzing the turning schedule of the patient by calculating a finite duration repositioning schedule based on a stress recovery model.

15. The method of claim 14 further comprising the step of:
developing an efficient and user-friendly summary of data derived from a plurality of pressure sensors, caregiver's data, processed data and risk assessment data.

16. A method for monitoring, prevention and management of pressure ulcers of a patient, the method comprising: acquiring at least one real-time physiological data stream; identifying a physiological data related to pressure present in the at least one real-time physiological data stream; selecting a first algorithm directed to produce a first diagnostic interpretation of the physiological data; selecting a second algorithm directed to produce a second diagnostic interpretation of the physiological data, wherein the first algorithm and the second algorithm produce diagnostic interpretations of the same identified physiological data; applying the first algorithm to the at least one real-time physiological data stream as the at least one real-time physiological data stream is acquired to produce at least one first diagnostic interpretation;
applying the second algorithm to the at least one real-time physiological data stream as the at least one real-time physiological data stream is acquired to produce at least one second diagnostic interpretation; and displaying the first and second diagnostic interpretations.

17. The method according to claim 16 further comprising the step of selecting a third algorithm directed to produce a third diagnostic interpretation of the physiological data; applying the third algorithm to the at least one real-time physiological data stream as the at least one real-time physiological data stream is acquired to produce a third diagnostic interpretation; and displaying the first, second and third diagnostic interpretations.

18. The method according to claim 17 further comprising the step of selecting a fourth algorithm directed to produce a fourth diagnostic interpretation of the physiological data; applying the fourth algorithm to the at least one real-time physiological data stream as the at least one real-time physiological data stream is acquired to produce a fourth diagnostic interpretation; and
displaying the first, second, third and fourth diagnostic interpretations.

19. The method according to claim 18 further comprising the step of selecting a fifth algorithm directed to produce a fifth diagnostic interpretation of the physiological data; applying the fifth algorithm to the at least one real-time physiological data stream as the at least one real-time physiological data stream is acquired to produce a fifth diagnostic interpretation; and displaying the first, second, third, fourth and fifth diagnostic interpretations.

20. The method according to claim 19 further comprising the step of selecting a sixth algorithm directed to produce a sixth diagnostic interpretation of the physiological data; applying the sixth algorithm to the at least one real-time physiological data stream as the at least one real-time physiological data stream is acquired to produce a sixth diagnostic interpretation; and displaying the first, second, third, fourth, fifth and sixth diagnostic interpretations.
